(19) United States
(12) Patent Application Publication
(10) Pub. No.: US 2017/0108484 A1
(43) Pub. Date: Apr. 20, 2017

(54) IDENTIFICATION OF BIOLOGICAL SAMPLES

(71) Applicant: Stephen Micheal HENRY, Auckland (NZ)

(72) Inventor: Stephen Micheal HENRY, Auckland (NZ)

(21) Appl. No.: 15/127,121

(22) PCT Filed: Mar. 18, 2015
(86) PCT No.: PCT/NZ2015/050031

§ 371 Date: Sep. 19, 2016

(30) Foreign Application Priority Data
Mar. 18, 2014 (NZ) 622647

(51) Int. Cl. G01N 33/483; G01N 33/49; G01N 33/493

(52) U.S. Cl. CPC G01N 33/4833; G01N 33/493; G01N 33/49

(57) ABSTRACT

Micro-dimensioned wafers for use in maintaining the traceability of biological samples are described.

### FIELD OF INVENTION

The invention relates to devices and methods for use in maintaining the traceability of biological samples. In particular, the invention relates to devices and methods for use in maintaining the traceability of embryos in assisted reproductive technologies (ART).

### BACKGROUND ART

The ability to confirm the chain of custody of biological samples is a critical requirement of forensic science, including anti-doping regimes. The ability to confirm the provenance of biological samples is also of increasing importance with the emergence of organised collections of human biological material and associated information (“biobanks”)(Caulfield et al (2014).

With the expansion in the application of human assisted reproductive technologies (ART) and in anticipation of the wider application of stem cell based therapies in regenerative medicine, ensuring the authenticity and traceability of biological samples is of increasing importance. In the application of ART the mismatching of biological samples (eggs and sperm) can be catastrophic for the parties concerned. The multiple steps involved in sourcing the biological samples, in vitro processing and administration to a human subject create numerous opportunities for inadvertent error or, in extreme circumstances, deliberate adulteration or falsification of records.

The publication of Arav et al (2004) discloses a method and system of controlling the processing of biological samples in which each component is assigned a unique machine readable identification mark. The method and system provides for the correct matching of the holders and containers of the biological samples by concurrent identification, but does not eliminate the risk of mismatching arising from errors when the biological samples are transferred to and from the holders and containers. In the disclosed method and system the holders and containers of the biological samples are identified, not the biological samples per se.

The publication of Treff et al (2010) discloses a minimally invasive system for embryo tracking by single nucleotide polymorphism microarray-based DNA fingerprinting of the first polar body. The system is directed primarily to tracking which embryos implant after multiple endometrium transfer (ET). The publications of Novo at al (2011 and 2014) disclose a method of tagging embryos with wheat germ agglutin lectin-biofunctionalised polysilicon barcodes of micrometric dimensions. The method was demonstrated to have no significant effect of post-vitrification survival and morphokinetic parameters of tagged embryos.

It is an object of the present invention to provide an improved method of maintaining the traceability of biological samples, or at least to provide the public with a useful choice.

### STATEMENT OF INVENTION

In a first aspect the invention provides a micro-dimensioned wafer provided with a plurality of peripherally spaced apart identifier indicia and a reference indicium.

Preferably, the micro-dimensioned wafer is fabricated from an inert material. More preferably, the micro-dimensioned wafer is fabricated from a polyimide.

Preferably, the surface of the micro-dimensioned wafer is biofunctionalised. More preferably, the surface of the micro-dimensioned wafer is biofunctionalised with avidin or a wheat germ agglutinin lectin. Most preferably, the surface of the micro-dimensioned wafer is biofunctionalised with avidin.

Preferably, the micro-dimensioned wafer is substantially circular.

Preferably, the micro-dimensioned wafer is asymmetric.

In a second aspect the invention provides a method of validating the source of a biological fluid comprising the steps of:
mixing the fluid with wafers of the first aspect of the invention to provide an identifiable sample; and subsequently
identifying the sample by determining the combination of the wafers present in the sample,

where the wafer has a pre-determined combination.

Preferably, the identifying is by an image processing system.

In a third aspect the invention provides a composition that is miscible with biological fluids and comprising one or more wafers of the first aspect of the invention.

In a first embodiment of the third aspect of the invention the composition is a solid tablet.

In a second embodiment of the third aspect of the invention the composition is a liquid suspension.

Preferably, the composition is opaque.

In a fourth aspect the invention provides a method of maintaining the traceability of a biological sample comprising the step of localising the biological sample to the surface of a biofunctionalised micro-dimensioned wafer of the first aspect of the invention.

Preferably, the biological sample is an embryo. More preferably, the biological sample is a human embryo.

Preferably, the biological sample has been biotinylated and the biofunctionalised micro-dimensioned wafer is an avidin functionalised micro-dimensioned wafer.

Preferably, the wafer is translucent.

In the description and claims of this specification the following acronyms, terms and phrases have the meaning provided: “asymmetric” means having no rotational axis of symmetry; “comprising” means “including”, “containing” or “characterized by” and does not exclude any additional element, ingredient or step; “consisting of” means excluding any element, ingredient or step not specified except for impurities and other incidentals; “hydrophilic” means having a tendency to be wetted by water; “localised” means associated with a surface by non-covalent interactions and “localising” and “localisation” have a corresponding meaning; “micro-dimensioned” means dimensions of 1 to 1,000 microns (μM); “miscible” means forming a homogenous mixture when added together; “substantially circular” means of circular shape when the presence of the peripherally spaced apart identifier indicia is disregarded and “translucent” means allowing light to pass through partially.

The terms “first”, “second”, “third”, etc. used with reference to elements, features or integers of the subject matter defined in the Statement of Invention and Claims, or when used with reference to alternative embodiments of the invention are not intended to imply an order of preference.

Where concentrations or ratios of reagents are specified the concentration or ratio specified is the initial concentration or ratio of the reagents.

Where values are expressed to one or more decimal places standard rounding applies. For example, 1.7 encompasses the range 1.650 recurring to 1.7499 recurring.

The invention will now be described with reference to embodiments or examples and the figures of the accompanying drawings pages.

### BRIEF DESCRIPTION OF DRAWINGS

FIG. 1. An embodiment of a micro-dimensioned, substantially circular wafer provided with a plurality of identifier indicia and an asymmetric reference indicium.

FIG. 2. A copy of FIG. 1 showing the parameters (rₙ, sₙ) usable in the programming of both the laser cutting apparatus used in the fabrication of the wafers and the image processing system used in their identification.

FIG. 3. An embodiment of a biofunctionalised micro-dimensioned, substantially circular wafer provided with a plurality of identifier indicia and an asymmetric reference indicium.

FIG. 4. Examples of the embodiment of the biofunctionalised micro-dimensioned, substantially circular wafer illustrated in FIG. 2 provided with an asymmetric reference indicium and different combinations of a plurality of identifier indicia.

FIG. 5. A schematic representation of a method of maintaining the traceability of an embryo using a wheat germ agglutinin lectin functionalised micro-dimensioned, substantially circular wafer.

FIG. 6. A schematic representation of a method of maintaining the traceability of a biotinylated embryo using an avidin functionalised (avidinylated) micro-dimensioned, substantially circular wafer.

FIG. 7. An artist's impression of an embryo localised to a biofunctionalised, micro-dimensioned, substantially circular wafer showing the relative dimensions of the biological sample (embryo) and the wafer.

### DETAILED DESCRIPTION

The combination of the locations of the plurality of peripherally spaced apart identifiers relative to the reference indicium serves to identify a wafer. The term “combination” is used in the claims and the remainder of this description to refer to the set of relative locations that serve to identify the wafer. Similarly micro-dimensioned, substantially circular wafers can be fabricated with a common reference indicium and different combinations. The similarly micro-dimensioned, substantially circular wafers are fabricated using laser cutting apparatus programmable to provide multiple copies of wafers with different combinations. These combinations are optically readable by image processing systems programmed to recognise the similarly micro-dimensioned, substantially circular wafers.

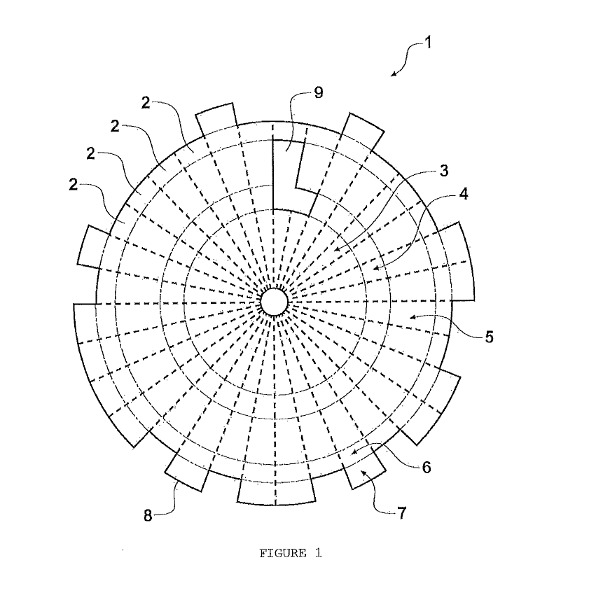

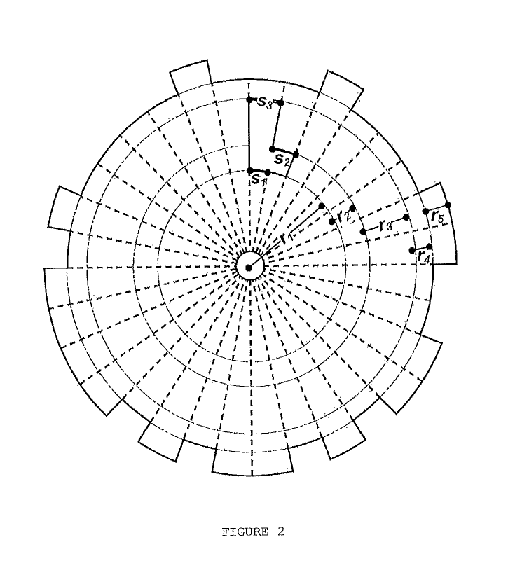

For the purpose of programming the laser cutting apparatus and the image processing system the micro-dimensioned, substantially circular wafer (1) is divided into a plurality of equal angular slices (2), each one of the angular slices being subdivided into common radial segments (3,4,5,6,7) as illustrated in FIG. 1. The dimensions employed as programming parameters are illustrated in FIG. 2.

An identifier indicium (8) is provided by the presence of the outer radial segment (7) of an angular slice of the micro-dimensioned, substantially circular wafer (1). The reference indicium (9) may be provided by the absence of an asymmetric arrangement of internal radial segments of one or more angular slices, such as the “L-shaped” void shown in FIG. 1.

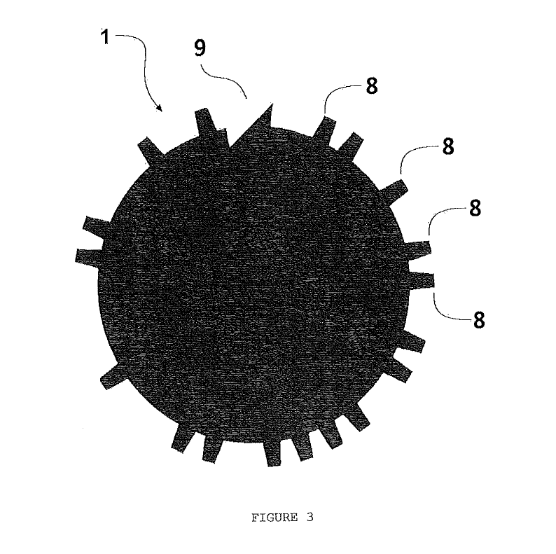

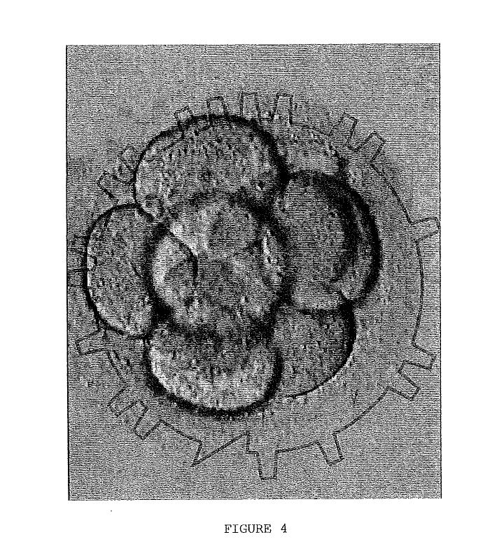

It will be recognised that in some embodiments of the wafer the reference indicium (9) is desirably located at or proximal to the periphery of the micro-dimensioned, substantially circular wafer (FIG. 3). An example of when such an embodiment is desirable is when a biological sample such as an embryo is localised to the surface of the micro-dimensioned, substantially circular wafer as illustrated in FIG. 4.

The wafers are fabricated from a resilient, inert material compatible with their addition to biological fluids and contacting with biological entities. Polyimide has been identified as a suitable material for use in the former circumstance. The trade name product KAPTON™ (DuPont) is an example of a polyimide film suitable for use in the fabrication of the micro-dimensioned, substantially circular wafers using laser cutting apparatus.

In a method of validating the source of a biological fluid such as blood or urine, wafers of a pre-determined combination, i.e. known to the receiver of the collected sample, are supplied to the donor of the sample. The wafers may be included in the collection vessel or supplied separately. When supplied separately the wafers are desirably supplied as a composition that can be conveniently mixed with the sample by the donor or their witness at the time of collection.

Advantageously, the composition is in the form of a tablet consisting of the wafers and excipients compatible with the biological fluid and subsequent analyses. Following dissolution of the tablet the wafers will be present in all aliquots of the collected sample taken by the receiver.

Any handling errors by the receiver are readily detected by a lack of correspondence between the pre-determined combination of the wafers supplied to the donor and the, identifiers used on the collection vessel and any other vessels into which aliquots of the collected sample are dispensed.

The wafer may also be biofunctionalised with avidin or wheat germ agglutinin lectin. Despite being inert, the surface of the wafer can conveniently be biofunctionalised by immersing in a dispersion of biotin-lipid conjugates as disclosed in the publication of Henry and Parker (2014).

The localisation of biotin (“biotinylation”) to the surface of embryos is conveniently achieved using the water dispersible functional lipid constructs described in the publication of Bovin at al (2009). The wafers can be dimensioned to promote localisation of a single embryo to each wafer. For example, for early stage embroys (blastocysts) a wafer with a diameter of circa 150 μM may be used. The wafer will typically have a thickness of only about 25 μM. A wafer that is translucent aids in the visualisation of the embryo when localised to the surface of the wafer.

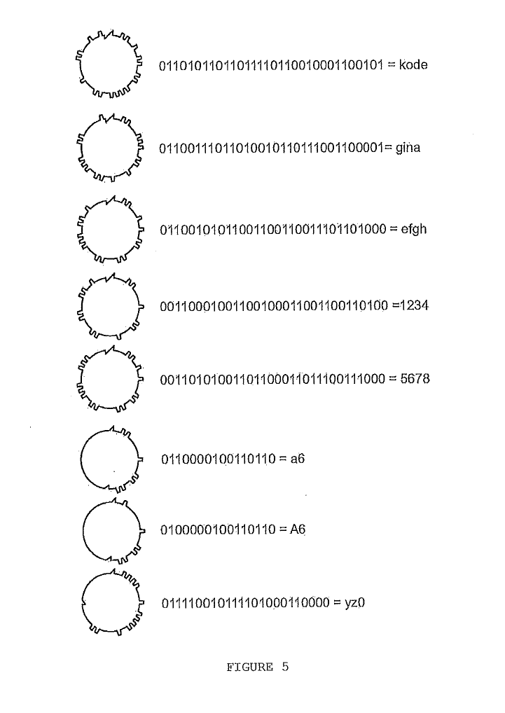

Examples of wafers that may be used in a method of maintaining the traceability of an embryo and the corresponding binary codes are illustrated in FIG. 5. Examples of assignable two to four character alphanumeric codes are also provided in FIG. 5.

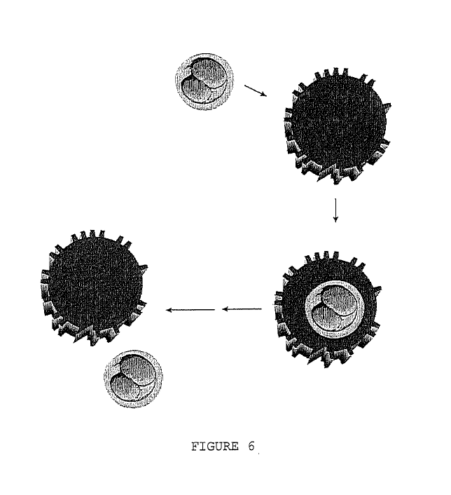

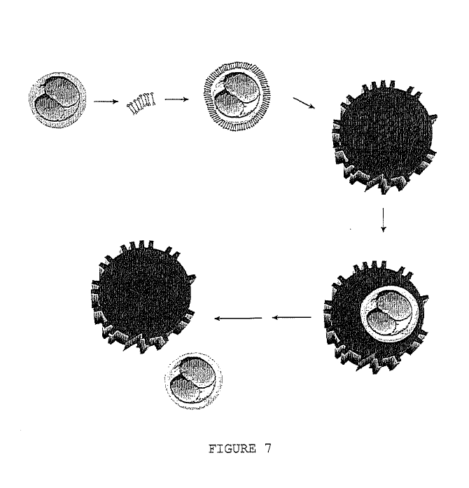

Methods of maintaining the traceability of an embryo employing biofunctionalised wafers are illustrated schematically in FIG. 6 and FIG. 7. As discussed above, the wafer may be biofunctionalised with either wheat germ agglutinin lection (FIG. 6) or avidin (FIG. 7). In the method illustrated schematically in FIG. 6 the embryo is biotinylated in a preliminary step before localisation to the surface of the wafer. It will be recognised that this localisation can be achieved simply by contacting the embryo with the wafer in media routinely used in ART thereby minimising the risk of harm to the developing embryo.

Although the invention has been described with reference to embodiments or examples it should be appreciated that variations and modifications may be made to these embodiments or examples without departing from the scope of the invention. Where known equivalents exist to specific elements, features or integers, such equivalents are incorporated as if specifically referred to in this specification. In particular, variations and modifications to the embodiments or examples that include elements, features or integers disclosed in and selected from the referenced publications are within the scope of the invention unless specifically disclaimed. The advantages provided by the invention and discussed in the description may be provided in the alternative or in combination in these different embodiments of the invention.

### REFERENCED PUBLICATIONS

Arav et al (2004) Method and system for controlling the development of biological entities international application no. PCT/IL2003/000544 (publ. no. WO 2004/003131 A2).

Bovin et al (2009) Functional Lipid Constructs International Application no. PCT/NZ2008/000266 (Publ. no. WO 2009/048343).

Caulfield et al (2014) A review of the key issues associated with the commercialisation of biobanks Journal of Law and the Biosciences, 94-110.

Henry and Parker (2014) Biocompatible method of functionalising substrates with inert surfaces International Application No. PCT/NZ2012/000242 [publ. no. WO 2014/007649].

Novo et al (2011) A novel embryo identification system by direct tagging of mouse embryos using silicon-based bar codes Human Reproduction, 26(1), 96-105.

Novo et al (2014) Bar code tagging of human oocytes and embryos to prevent mix-ups in assisted reproduction technologies Human Reproduction, 29(1), 18-28.

Treff at al (2010) Robust embryo identification using first polar body single nucleotide polymorphism microarray-based DNA fingerprinting Fertility and Sterility, 93(7), 2453-2455.

1)-6) (canceled)

7) A micro-dimensioned substantially circular wafer provided with a plurality of peripherally spaced apart identifier indicia and a reference indicium where the combination of the locations of the plurality of peripherally spaced apart identifier indicia relative to the reference indicium serves to identify the wafer.

8) The wafer of claim 7 where the surface is biofunctionalised.

9) The wafer of claim 8 fabricated from an inert material.

10) The wafer of claim 9 fabricated from a polyimide.

11) The wafer of claim 10 where the surface is avidinylated.

12) A method of validating the source of a biological fluid comprising the steps of:
mixing the fluid with a plurality of wafers of claim 7 to provide an identifiable sample; and subsequently
identifying the sample by determining the identity of the wafers present in the sample,
where the wafers have a pre-determined identity.

13) The method of claim 12 where the biological fluid is blood or urine.

14) The method of claim 13 where the identifying is by an image processing system.

15) A method of maintaining the traceability of a biological entity comprising the step of localising the entity to the surface of a wafer of claim 11.

16) The method of claim 15 where the entity has been biotinylated.

17) The method of claim 16 where the entity is an embryo.
